(19) United States
(12) Patent Application Publication
(10) Pub. No.: US 2007/0239059 A1
(43) Pub. Date: Oct. 11, 2007

(54) Neurophysiology testing system

(76) Inventor: Christopher R. McIver, Checotah, OK (US)

Correspondence Address: LITMAN LAW OFFICES, LTD., P.O. BOX 15035, CRYSTAL CITY STATION, ARLINGTON, VA, 22215, US

(21) Appl. No.: 11/650,426

(22) Filed: Jan. 8, 2007

Related U.S. Application Data
(60) Provisional application No. 60/783,836, filed on Mar. 21, 2006.

(51) Int. Cl. A61B 5/04

(52) U.S. Cl. 600/544; 600/383; 600/386; 600/545

(57) ABSTRACT

The neurophysiology testing and monitoring system provides a multifunctional neurological testing system for a variety of neurological tests, such as electroencephalographs (EEGs) and magnetoencephalographs (MEGs), to be performed on a patient. The system includes a housing for receiving at least one neurological measurement system and a conductive gel supply. A selectively positionable elongated support is pivotally mounted on an upper end of the housing at a proximal end thereof, and a sensor cap is mounted on the distal end of the elongated support. The sensor cap is selectively and adjustably positioned to be adjacent the head of the user so that at least one sensor, such as an EEG electrode, mounted on the sensor cap comes into contact with the patient's head. The EEG electrode is in communication with the conductive gel supply for selective application of conductive gel to the user's head.

### CROSS-REFERENCE TO RELATED APPLICATION

This application claims the benefit of U.S. Provisional Patent Application Ser. No. 60/783,836, filed Mar. 21, 2006.

### BACKGROUND OF THE INVENTION

1. Field of the Invention

The present invention relates to neurological testing devices, and particularly to a neurophysiology testing and monitoring system for making a variety of neurological measurements.

2. Description of the Related Art

Neurophysiological testing and monitoring often requires a wide variety of equipment, such as encephalographic (EEG) measurement systems, computers, sensor assemblies, signal amplifiers, pre-amplifiers and the like, which are typically assembled in a research laboratory, a doctor's office or hospital room, with the various components being arranged around the patient. Typically, each separate component is purchased and assembled separate from the other components, and the various components are arranged and interconnected by medical practitioners and scientists.

As a result, neurological testing facilities tend to be cluttered, which can be dangerous in an emergency medical setting, and the equipment is often connected in a makeshift manner by technicians who are not well trained in electronics, computers, and other related fields. Thus, it would be desirable to provide a single system containing the necessary equipment for a wide variety of neurological testing, which would be both space-efficient and would not require assembly or interconnection of a wide variety of separately purchased and manufactured equipment.

Neurophysiological testing, such as EEGs and MEGs, often involves the use of a harness with attached sensors for application to the patient's head. Typical harnesses are uncomfortable, cumbersome and can be overly heavy, thus causing severe discomfort for the patient. Additionally, conventional harnesses are unsupported, and portions of the harness, and the associated wires and cables, often lie on the floor of the room and across the work surfaces of the neurological testing equipment, which can be dangerous and can cause electrical errors in the measurements. It would be desirable to provide a supported harness, both for the comfort of the user and in order to prevent the hazards associated with the harness and associated wires and cables resting on the floor and other surfaces.

Heavier harnesses have been supported from the ceiling of the room, typically in medical research environments, in order to prevent injury and discomfort to the user. However, such harnesses are relatively immobile and can only be lowered and raised from the ceiling. In order to allow free positioning of the patient, it would be desirable to provide a supported harness which may be adjustably and selectively positioned to be positioned adjacent the user's head.

Typically, the harnesses include an attached electrode cap, which is releasably attached to the user's head and includes one or more sensors, such as EEG electrodes, for example. Commonly used electrode caps include two or three pieces of fabric, formed from a comfortable and resilient material, such as a Lycra® (spandex). Such electrode caps, however, typically have seams which interfere with the placement of electrodes and can cause interruption in the signal transmission between the scalp of the patient and the electrodes located near them.

These fabric seams become even more problematic as the number of electrodes embedded in the cap increases. Typical electrode caps include approximately nineteen to forty electrode contact points, from which the diagnostician diagnoses and monitors neurological activity throughout the brain of the user. Electrode caps may include up to two hundred and fifty-six contact points, with the fabric of typical caps causing even greater interference problems. It would be desirable to provide an electrode cap formed without interfering seams in order to effectively apply electrodes to the user's head.

In typical EEG practice, a medical technician positions the electrode cap on the patient's head and applies conductive gel between each electrode and the patient's scalp. The technician then, through application of electrical current to the electrodes, determines whether each electrode is in proper electrical contact with the patient's scalp. If so, the electrical signals received are recorded for subsequent interpretation by a neurologist, researcher, or other medical practitioner.

Conventional electrodes used with the EEG methodology include a contour to form an electrode cup, which is then manually filled with the conductive gel, prior to performing the encephalographic study. This manual filling process is time consuming, laborious and messy. Typical gel filling systems include a blunted needle attached to a gel-filled syringe. The technician passes the needle through an opening formed through the electrode and injects the conductive gel into the electrode cup, which is fastened to the cap and is positioned against the client's scalp. The blunted needle is further used to abrade the user's scalp, if necessary, to reduce the input impedance to acceptable levels. At a manual filling rate of approximately one minute per electrode cup, it would take a single technician over four hours to fill an electrode cap containing two hundred fifty-six electrodes. It would be desirable to provide a pressurized or automated system for the controlled filling of the electrode cups. As multimodal physiologic data is gathered from a patient, it would be further desirable to visualize the information and correlate it to the user's anatomy.

Thus, a neurophysiology testing and monitoring system solving some or all of the aforementioned problems is desired.

### SUMMARY OF THE INVENTION

The neurophysiology testing and monitoring system provides a multifunctional neurological testing and monitoring system for a variety of neurological studies, such as electroencephalographs (EEGs), spinal cord monitoring and peripheral nerve testing, and magnetoencephalographs (MEGs), to be performed on a patient. The system includes a housing for receiving at least one neurological measurement system and a conductive gel supply. A selectively positionable elongated support is pivotally mounted on an upper end of the housing, at a distal end thereof, and a sensor cap is mounted on the proximal end of the elongated support. The elongated support may include a counterweight mounted on the distal end thereof for manual positioning of the elongated support or, alternatively, a motor-driven assembly may be mounted on the housing for automatic control of positioning of the elongated support.

The sensor cap is selectively and adjustably positioned to be adjacent the head of the user, so that at least one sensor mounted on the sensor cap, such as an EEG electrode, comes into contact with the patient's head. The EEG electrode is in communication with the conductive gel supply for selective application of conductive gel to the user's scalp. The electrode includes an electrode cup having an open lower end for placement on the user's head. An elongated shaft is mounted to the upper end of the electrode cup and has a hollow chamber defined therein, which is in fluid communication with an interior region of the electrode cup. A gel port is formed through a sidewall of the elongated shaft for connection with the conductive gel supply.

A dispensing system, such as a pump, may be mounted on the housing for selective dispensing of conductive gel from the conductive gel supply to the electrode for application to the user's head. Additionally, the upper end of the elongated shaft may be open and adapted for receiving an abrading instrument. The abrading instrument is received within the shaft and within the electrode cup for abrading the scalp of the user prior to application of the conductive gel.

These and other features of the present invention will become readily apparent upon further review of the following specification and drawings.

### BRIEF DESCRIPTION OF THE DRAWINGS

FIG. 1 is an environmental, perspective view of a neurophysiology testing system according to the present invention, partially broken away to show details of the invention.

FIG. 2 is a block diagram of a motion control unit of the neurophysiology testing system according to the present invention.

FIG. 3 is a block diagram of measurement unit of the neurophysiology testing system according to the present invention.

FIG. 4 is a block diagram of a sleep studies measurement unit of the neurophysiology testing system according to the present invention.

FIG. 5 is a block diagram of an electroencephalograph (EEG) measurement unit of the neurophysiology testing system according to the present invention.

FIG. 6 is a block diagram view of a gel pump unit of the neurophysiology testing system according to the present invention.

FIG. 7 is a block diagram of a magnetoencephalograph (MEG) unit of the neurophysiology testing system according to the present invention.

FIG. 8 is a diagrammatic view of an EEG electrode system of the neurophysiology testing system according to the present invention.

FIG. 9 is a diagrammatic view of an MEG electrode system of the neurophysiology testing system according to the present invention.

FIG. 10 is a side view in section of an electrode of the neurophysiology testing system according to the present invention.

FIG. 11 is a side view in section of an MEG sensor of the neurophysiology testing system according to the present invention.

FIG. 12 is an environmental side view of an electrode cap of the neurophysiology testing system according to the present invention.

FIG. 13 is a diagrammatic view of an alternative embodiment of the neurophysiology testing system according to the present invention.

FIG. 14 is a perspective view of an alternative embodiment of the neurophysiology testing system according to the present invention.

FIG. 15 is a partially cut-away, perspective view of an alternative embodiment of an electrode used in combination with the neurophysiology testing system according to the present invention.

Similar reference characters denote corresponding features consistently throughout the attached drawings.

### DETAILED DESCRIPTION OF THE PREFERRED EMBODIMENTS

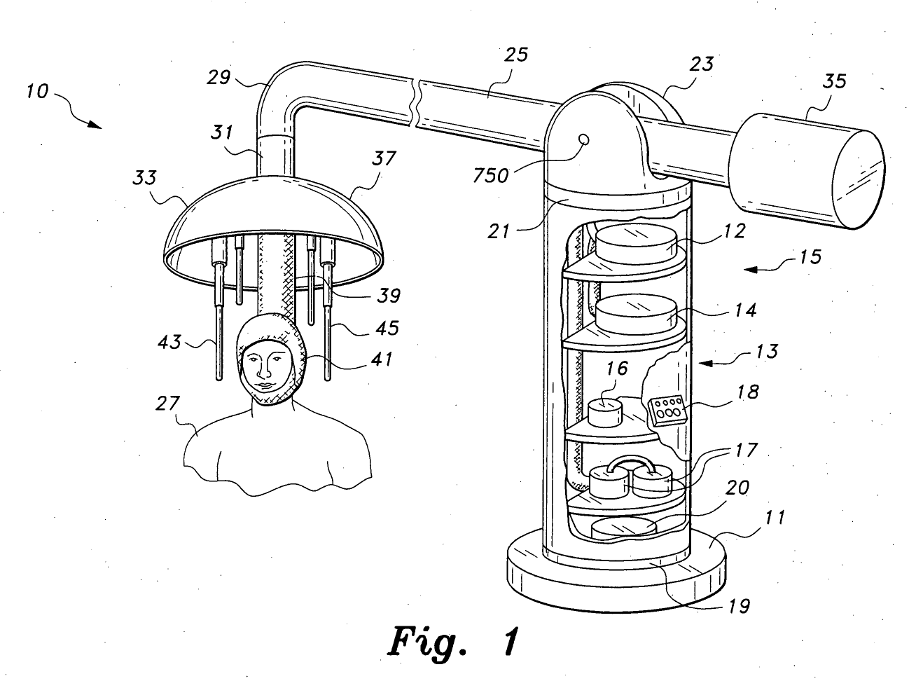

As shown in FIG. 1, the present invention is directed towards a neurophysiology testing system, which provides a multifunctional neurological testing system for a variety of neurological tests to be performed on a patient. The system, referred to generally as 10 in the drawings, includes a housing 13 for receiving at least one neurological measurement system 15 and a conductive gel supply 17.

Housing 13 may receive a wide variety of neurological testing equipment, such as that associated with EEGs, MEGs, surface and needle electromyography (EMG), motor and sensory nerve conduction tests (NCV), somatosensory evoked potentials (SSEP), repetitive nerve stimulation studies (RNS), brainstem auditory evoked responses (BAERs), visual evoked potentials (VEPs), polysomnography (PSG), or any other suitable or desired studies associated with neuroscience practice and research. Measurement system 15 may further include standardized encephalographic systems that perform very low, low, intermediate, and/or high sensor density encephalographic studies, which produce a functional organizational map of the brain with a high temporal resolution and an incrementally higher spatial resolution.

Further, the neurophysiological data gathered with measurement systems 15, disposed within housing 13, may be integrated with multiple external sources of image data, such as vascular sonographic studies, magnetic resonance imaging (MRI), functional MRI (fMRI), computed tomography scanning (CT), positron emission tomography (PET), or single photon emission computed tomography (SPECT).

As shown in FIG. 1, system 10 includes housing 13, which has an upper end and a lower end. A base 11 is mounted on the lower end of housing 13, for supporting housing 13 on a support surface, such as a floor. A lower surface of the base 11 may include a plurality of vertically adjustable countersunk castors, allowing the housing 13 to be easily transported, and allowing the system 10 to be easily positioned relative to a patient.

The size and contouring of housing 13 may vary according to the type and amount of measurement systems housed therein. However, in the preferred embodiment, the housing 13 is substantially cylindrical in contour, having a height of approximately two meters and having a diameter of preferably less than one meter. The open, interior region of housing 13 (shown in the broken away view of FIG. 1) may be divided into a plurality of compartments for containing measurement systems 15 and associated components of the testing system 10. For example, housing 13 may further contain an EEG pre-amplifier 12; an EEG amplifier 14; a 3-D electrode digitizer system electronic control unit 16; MEG hardware and peripherals; hardware, software and peripherals associated with the performance of desired nerve studies, including, but not limited to, electromyographic (EMG) studies, somatosensory studies, sensory and motor nerve conduction studies; late response studies (for example, F-wave and H-reflex studies); RNS studies; as well as any other desired nervous system studies typically performed in a neurology clinic, laboratory or operating room. The housing 13 may further contain a medical grade power supply unit 20.

The housing 13 may further contain a gel supply unit 17 and an automated motion control system unit 18, both of which will be described below in further detail. Each compartment of housing 13 may include a respective set of doors supported by countersunk rivets, handles and latches, or other suitable devices in order to maintain the substantially cylindrical contour of housing 13.

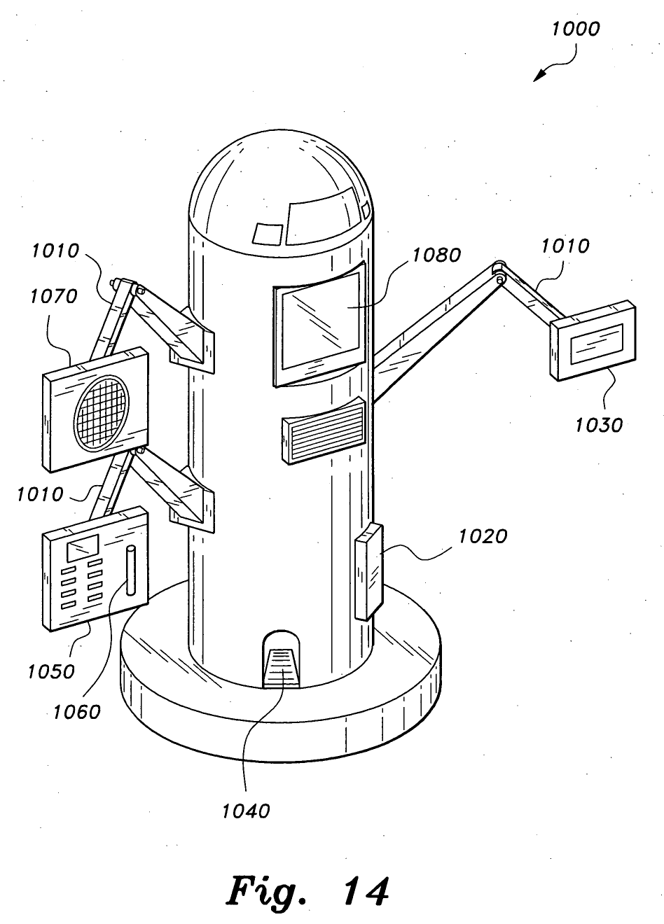

In an alternative clinical embodiment, illustrated in FIG. 14, system 1000 includes a plurality of extensible, adjustable pivoting arms 1010 for holding a variety of user-selectable equipment. In the exemplary embodiment of FIG. 14, the arms 1010 are shown supporting a photic stimulator 1030, an EP/EMG headbox 1050 (along with associated stimulator 1060), and an EEG headbox 1070, respectively. Further, the system may be at least partially operated through use of an attached foot pedal actuator 1040. The system may further include a visual monitor 1080, a keyboard and a printer 1020.

The system may further include an audio pickup, such as a microphone or the like, for receiving voice commands from the user. Further, infrared sensors or any other suitable video recording devices may be incorporated into the system, allowing at least one part of the system, such as one extensible arm, to physically follow the user under automatic, computerized control. On voice command, the system may navigate through laboratories, hallways, rooms and the like for testing and monitoring hospital inpatients, and patients in the intensive care unit (ICU). In a further alternative embodiment, the simultaneous multimodal testing and monitoring unit may be housed within an 8 inch by 10 inch (for example) base unit configured with a laptop computer for improved portability.

Returning to system 10, a disc turntable 19 may be sandwiched between the upper surface of base 11 and the lower surface of housing 13, as shown in FIG. 1. Preferably, turntable 19 provides approximately 120° rotation of housing 13 and is lockable, allowing the housing 13 to be angularly adjusted for use with a fixed patient. Similarly, a lazy Susan-type carousel 21 is preferably mounted on the upper surface of housing 13, allowing for rotation of bifurcated dome 23, to be described in greater detail below. Carousel 21 preferably offers approximately 120° of rotation and may also be locked into a fixed angular position.

Preferably, carousel 21 is formed from a fiberglass-reinforced nylon material, although it may be formed from any suitable material. Preferably, carousel 21 includes an internal gear system and/or bearing on its lower end, allowing for the locking rotation of bifurcated dome 23 with respect to housing 13. This rotation may be automated, as will be described in detail below.

In the embodiment of FIG. 1, a boom or elongated support 25 is further provided, having opposed proximal and distal ends. The proximal end of support 25 is pivotally mounted within a slot formed in dome 23 by a pivot pin 750 or the like. The proximal end may further include a geared mounting to the dome 23 to provide approximately 60° rotation of the elongated support 25 with respect to bifurcated dome 23. This rotation may be manually or automatically controlled, as will be described in further detail below.

The elongated support 25 preferably has a substantially cylindrical contour, which, in the preferred embodiment, has a circumference of approximately thirty centimeters and is approximately two to three meters in length. The elongated support 25 allows for testing on the patient with the patient being spaced apart from housing 13, reducing feelings of claustrophobia and anxiety common in neurological testing.

A neck portion 29 is formed on the distal end of support 25 and extends downwardly to support a crown 33, which is joined to the neck portion 29 by a multiaxial joint 31. The multiaxial joint 31 includes gears that provide for automated motion along a plurality of axes. A counterweight 35 is mounted on the proximal end of the support 25 for maintaining the support 25 in a desired angular position.

As shown, crown 33 includes an open interior region 37 for receiving a harness 39. Only the distal end of harness 39 is illustrated in FIG. 1. Harness 39 extends along the length of support 25, preferably housed within a channel formed therein, and the proximal end of harness 39 is received within housing 13. As will be described in further detail below, the harness 39 supports an electrode cap 41, which may be housed within crown 33 during periods when the cap 41 is not in use. Support 25 may also house the necessary wiring and cables associated with use of electrode cap 41 and/or equipment used for other studies. Further, a light'source may be received within interior region 37 of crown 33, providing either additional illumination of the patient, or replacing the need for exterior lighting, such as lighting mounted on the ceiling of the room.

As shown, at least one extendible arm 43 is mounted on the inner surface of crown 33 and extends downwardly therefrom. Extendible arm 43 may support, for example, a photic stimulator, which is a device commonly used during clinical encephalographic studies. A similar extendible arm 45 may be mounted on the inner surface of crown 33 to extend downwardly to a position on the opposite side of the patient's head (the patient is referred to generally as 27 in the Figures). Arm 45 may support transmitter components of a 3-D electrode location digitizer or other components. Further, supports for other peripheral equipment, such as handheld stimulators used for nerve conduction studies, needle EMG cables, 3-D electrode location digitizer receivers and styli, audiometric quality insert earphones, and a manual response keypad may also be housed within crown 33 and housing 13. Crown 33, housing 13, and support 25 are preferably formed from nonconductive plastic materials, such as nylon, polyethylene, acrylic styrene acrylonitrile (ASA) or any other suitable material.

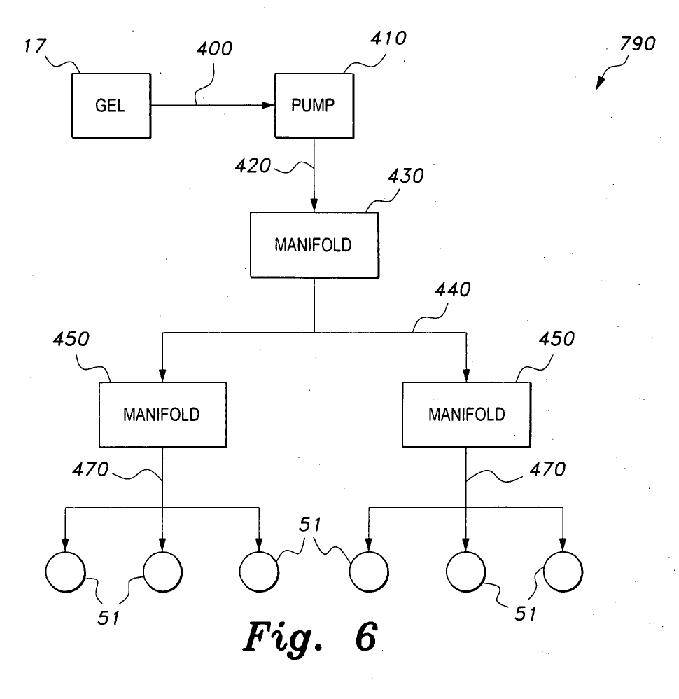

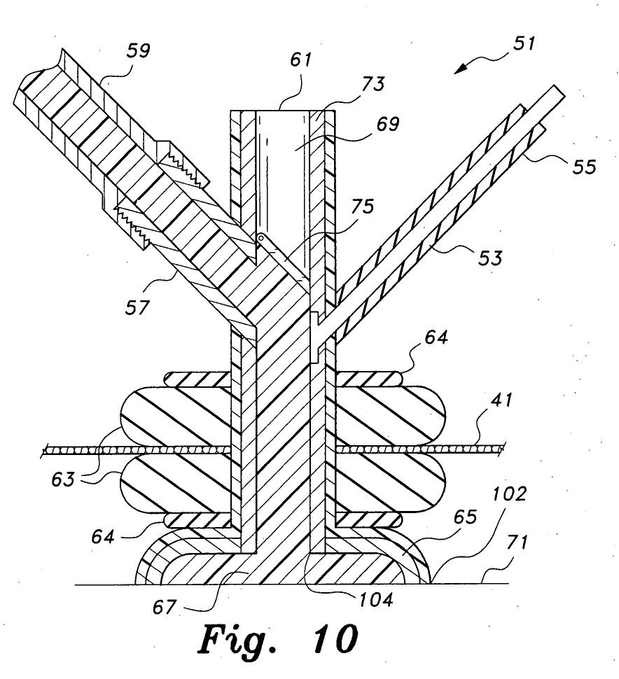

Cap 41 includes a plurality of electrode assemblies 51 (as will be described in greater detail below, with specific reference to FIG. 10). Conductive gel is injected into the electrode cup of each electrode 51 to provide a conductive electrical path between the electrode and the patient's scalp. System 10 injects the conductive gel from gel supply 17 into the multiple electrodes 51 simultaneously. The gel supply unit 17 includes a control subsystem for the application of the conductive gel prior to EEG testing, for example. As will be described in further detail below, with specific reference to FIG. 6, the gel supply unit 17 includes a supply of conductive gel, at least one pump, tubing and manifolds for the delivery of the conductive gel to the electrodes.

Once the electrode cap 41 has been placed on the patient's head, the gel pump is activated to fill a plurality of preselected electrode cups simultaneously. The gel is pumped through hoses 59 into harness 39.

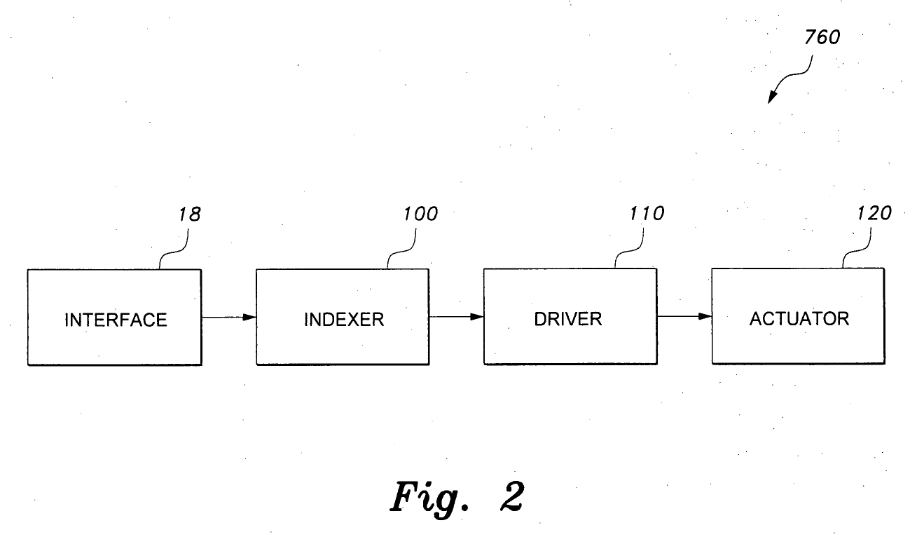

As shown in FIG. 2, the system 10 may further include an automated motion control unit 760 mounted on housing 13 for the control of support 25. The motion control unit 760 includes a user interface 18, which feeds user input to a microprocessor-based indexer 100, preferably with stand-alone, integrated and multiaxial control capabilities. A driver 110, which may be a bipolar chopper driver or any other suitable driver capable of converting indexer signals into suitable power to energize suitable actuators, is in electrical communication with indexer 100 and delivers the control signal to actuator 120, which may be a high inductance, microstepping, bifilar, hybrid stepping motor or the like. Actuator 120 rotates and positions support 25 under manual user control through user interface 18, or through preprogrammed commands.

In the preferred embodiment, the user interface 18 includes a countersunk motion control panel, including user-operated buttons and joysticks, mounted on housing 13. Interface 18 allows the user to rotate and selectively fix the housing 13 in the horizontal plane; raise, lower and fix the support 25 in the vertical plane; and rotate and fix the crown 33 along multiple planes about multiaxial joint 31. One such exemplary motion control unit is manufactured by the American Robotics® Corp. of Oakdale, Pa.

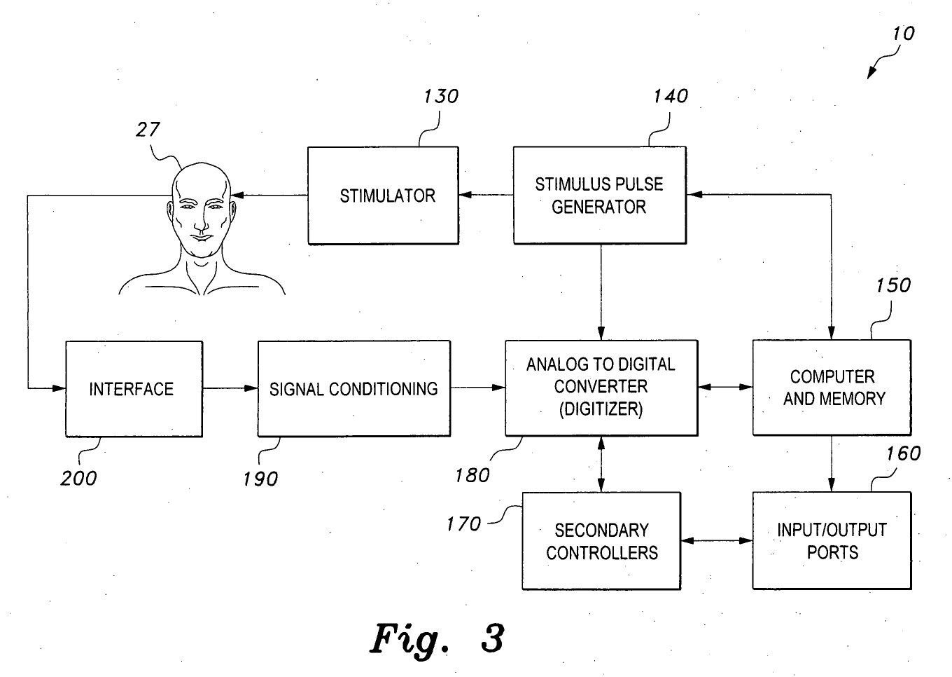

In the embodiment illustrated in FIG. 3, system 10 is equipped to perform nerve conduction studies (NCS), EMG and evoked potentials (EP). The system preferably includes electrodes, electrode leads, conductive paste, a handheld stimulator 130, a temperature probe, a foot switch, a control module, a headbox, a computer and associated memory 150, and suitable associated hardware and software. The handheld stimulator 130, electrode leads, temperature probe and needles may be housed in a crown 33 or housing 13.

As shown, user interface is performed through input/output ports 160, which may include a conventional user interface, such as that associated with a personal computer, or may include specialized instruments and displays. Similarly, automated control may be input through secondary controllers 170. Computer 160 generates a signal for actuation of stimulus pulse generator 140, which causes stimulator 130 to deliver a signal to the patient 27. It should be understood that the stimulator may, alternatively, be remotely located.

Interface 200, which may be in the form of electrodes 51, receives electrical signals generated by the patient 27, and the signals are received and translated by signal conditioning system 190. An analog-to-digital converter 180 may be employed to feed the signals back into computer 150 for subsequent generation of further stimulation signals.

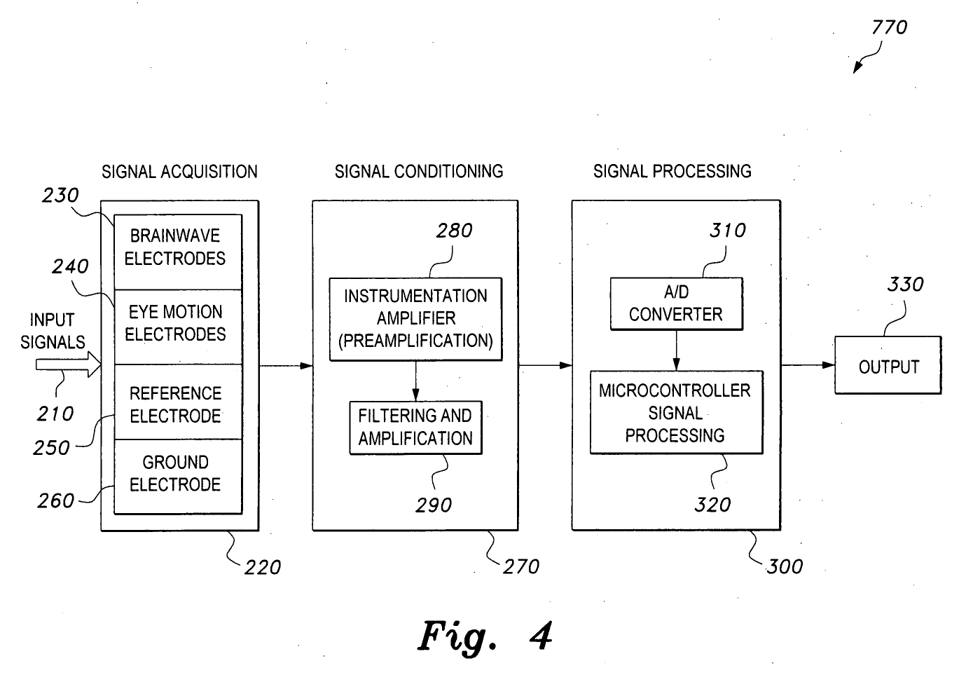

In the embodiment of FIG. 4, the system 10 may include polysomnogram (PSG) measuring equipment 770 so that the electrical signals 210 generated by the patient 27 are input into signal acquisition interface 220, which may include brainwave electrodes 230, eye motion electrodes 240, reference electrodes 250, ground electrode 260, body position sensors, cannulae, periodic limb sensors, respiratory effect sensors, snore sensors, pulse oximetry and the like, altogether referred to herein as PSG equipment. The signal conditioning subsystem 270 includes a pre-amplifier 280 and a filter/amplifier combination 290, which feeds the filtered and amplified signal to the signal processor 300. The signal processor 300 includes analog-to-digital converter 310 and microcontroller signal processor 320. The processed signal is then output at 330 for display to the operator and/or to be fed back into the control computer (160 of FIG. 3).

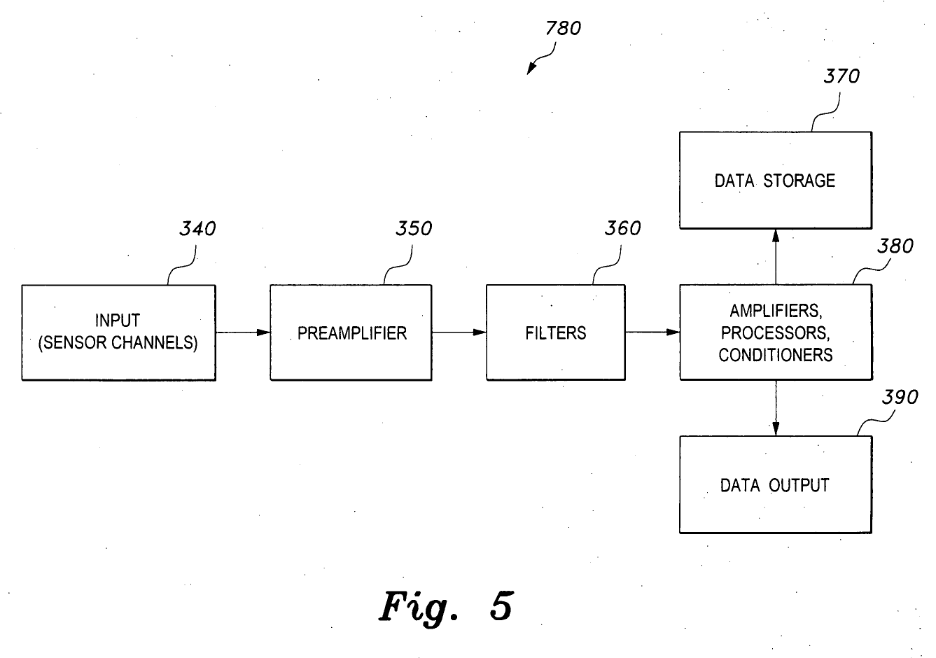

In the embodiment of FIG. 5, EEG measurement system 780 receives EEG input from the patient at 340, via the various selected sensor channels. These signals are delivered to pre-amplifier 350, and then filtered at 360. The signal is amplified at 380, stored at 370 and may be further conditioned and processed, dependent upon user-selected parameters. The resultant output signal may be displayed to the user at 390 and may also be stored for later retrieval at 370.

With reference to FIG. 6, a gel pumping subsystem 790 is illustrated. The subsystem 790 includes the conductive gel supply 17, which feeds into pump 410 via tubing 400. The pump delivers pressurized conductive gel to manifold 430 via tubing 420, which delivers the conductive gel via tubing 440 to secondary manifolds 450. The secondary manifolds 450 deliver the gel via tubing 470 to subsets of electrodes 51. The user may select from a variety of delivery options, such as the filling of only certain subsets of electrodes or individual electrodes.

Gel pump 410 is preferably automated and computer-controlled. Preferably, gel pump 410 is preprogrammable to deliver the conductive gel into preselected groups of electrodes, or into individual electrodes. Further, via user interface 18, the user preferably controls activation and deactivation of the pumping, and may manually adjust the flow rate of the conductive gel. A display may be provided, allowing the user to monitor the rate of gel injection, the volume of gel, and the elapsed time of the injection process.

Preferably, the initial phase of injection is preset to deliver conductive gel at a relatively moderate rate in order to minimize the time spent filling the electrodes of cap 41. A second phase initiates automatically at each manifold and continues to deliver gel at a controlled rate, as during the initial phase. Once the gel reaches the assigned electrodes, the change in pressure is sensed by the computer software in combination with suitable pressure sensors, allowing the electrodes to be injected with a precise amount of gel required to fill each electrode. The harness 39 may be constructed to provide visibility to the operator of the gel flow from the crown 33 to the cap 41. Alternatively, gel supply unit 17 may be portable and may be positioned separate from, and external to, housing 13.

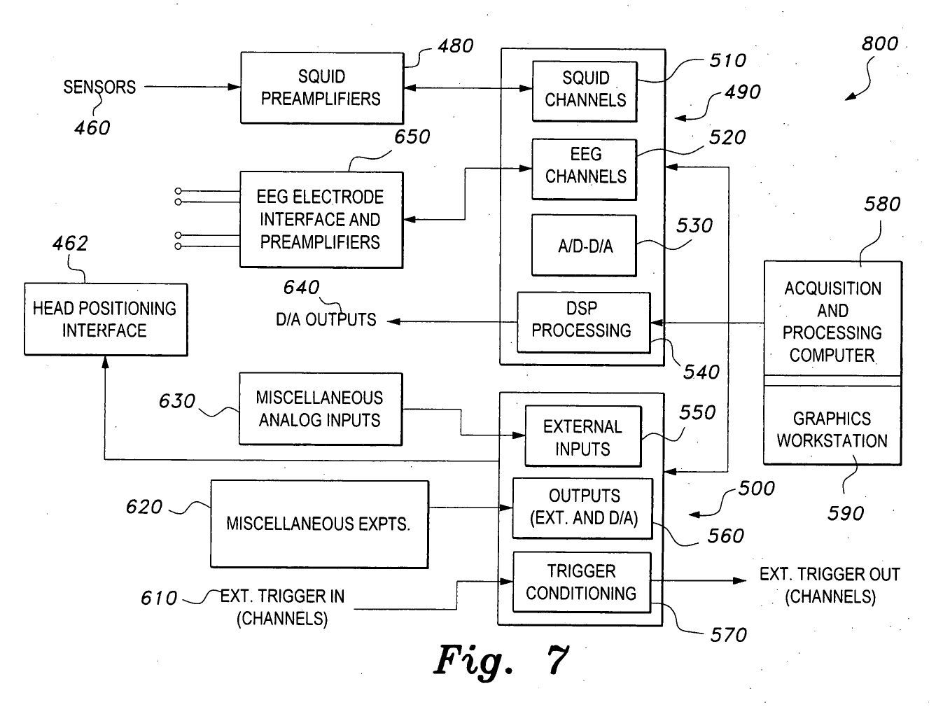

In the embodiment illustrated in FIG. 7, the system may include MEG equipment 800, including MEG sensors 460, which feed input signals from the patient to SQUID preamplifiers 480. The MEG system may be used in combination with an EEG system, including EEG electrodes and pre-amplifiers 650. The preamplified signals are transmitted to MEG/EEG electronics 490, which receive the SQUID channels 510, the EEG channels 520 and include analog-to-digital and digital-to-analog converters 530, as well as a DSP processor 540. The MEG/EEG electronics 490 are in communication with a peripheral interface unit 500, which receives external inputs 550 from selected analog peripherals 630, and includes an output system 560, which may be used to control a head positioning interface 462, and may further deliver a variety of exported signals, for display, etc. at 620. An external trigger 610 may be provided, with the triggering signals being conditioned and received at 570. External trigger signals are output at 600, and an SCSI interface transmits the processed signal from EEG/MEG electronics 490 to computer processor 580 and associated display 590.

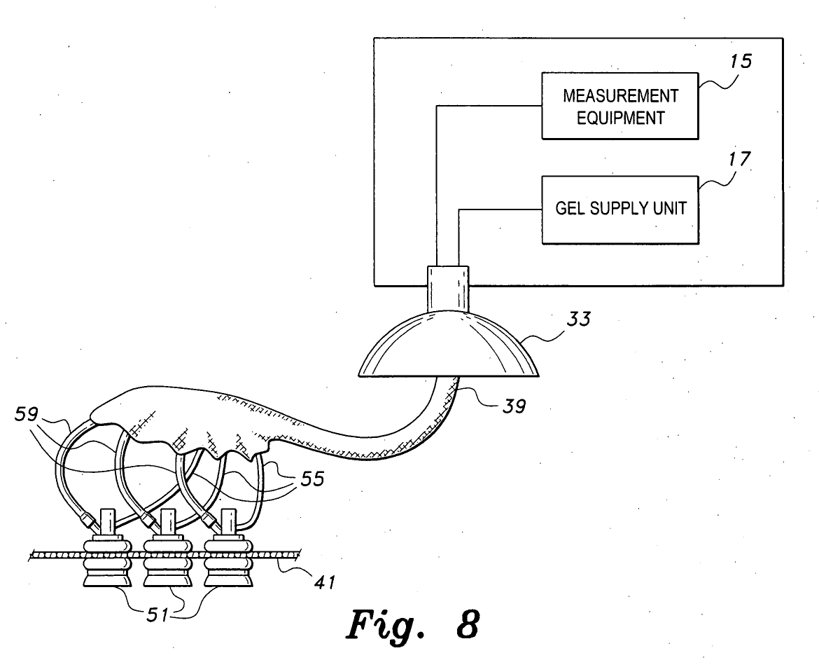

FIG. 8 shows a plurality of EEG electrode assemblies 51 mounted on cap 41. Electrode leads 55 are bundled within harness 39 and extend to measurement equipment 15. Gel tubing 59 is similarly bundled within harness 39 and extends to the gel supply unit 17. The individual bundles or subsets of electrodes 51 may include thirty-two, sixty-four, one hundred twenty-eight, two hundred fifty-six or five hundred twelve electrodes per subset. The associated gel tubing 59 for each bundle extends from the crown 33 to each assigned electrode 51 on cap 41. Preferably, the cap 41 includes approximately ten bundles or subsets of electrodes. In the preferred embodiment, each gel tube 59 is numbered and/or color-coded to match the number and/or color associated with particular electrode or electrode bundle.

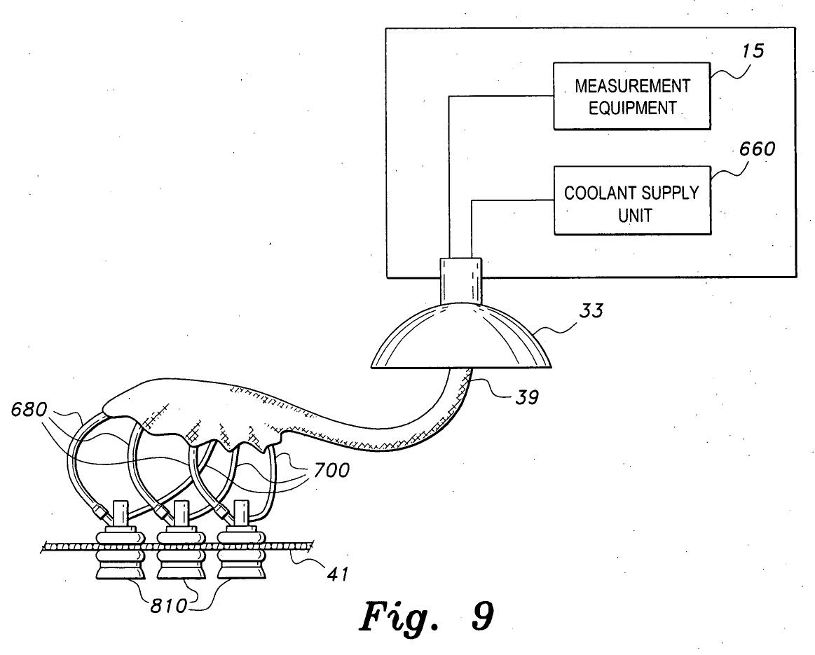

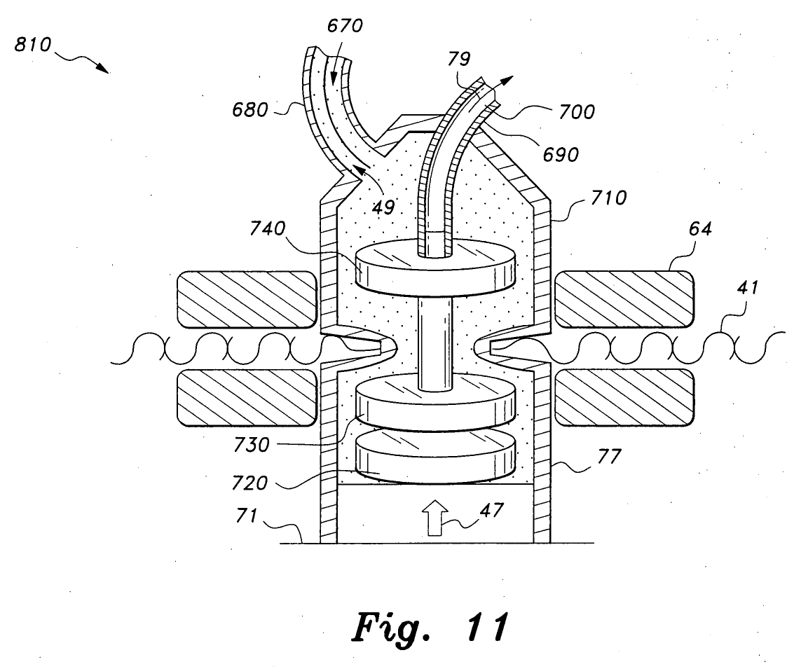

FIG. 9 shows an MEG sensor assembly. Rather than a conductive gel supply 17, the MEG system includes a coolant supply unit 660 for delivering coolant to MEG sensors 810 via tubing 680. The MEG sensors 810 will be described in greater detail below, with specific reference to FIG. 11. Similarly, each MEG sensor 810 has an electrical lead 700 associated therewith, which are bundled together within harness 39.

FIG. 10 illustrates a preferred embodiment of an electrode assembly 51 to be used with system 10. Each electrode 51 includes an electrode cup 65, which includes an open interior region 67 for receiving the conductive gel. The lower edge 102 of the electrode cup 65 is placed against the scalp 71 of the patient. Alternatively, this lower edge may be preloaded with a heat or fluid-activated conductive substance, and be protected with cellophane, plastic or the like, providing a disposable peel-away sheet located within the disposable electrode cap.

An opening 104 is formed through the upper surface of electrode cup 65 and a shaft 73 is mounted on the upper surface of cup 65 so that an internal passage 69 of shaft 73 is in communication with the interior region 67 via opening 104.

A connector 53 is mounted on a sidewall of shaft 73 for the mounting of electrical lead 55. A gel port 57 is further formed on the sidewall of shaft 73 for connection to gel tubing 59. As shown, the gel port 57 may include a threaded exterior surface for releasable mating with the tubing 59. Alternatively, any other suitable releasable fixture may be utilized.

An opening 61 is formed through the upper end of shaft 73 so that an abrading tool may be inserted through opening 61, passed through passage 69 and opening 104 and received within region 67 to abrade the scalp 71 of the patient. A pivoted flapper valve 75 is mounted within passage 69, as shown, to allow insertion of the abrading tool, but providing a seal when conductive gel is charged into the electrode assembly, so that the conductive gel does not escape from the electrode via opening 61. The valve 75 may have an opening formed therethrough for return air flow when the gel pump is activated.

Each electrode 51 is mounted on a very low density (approximately thirty-two electrodes), low density (approximately thirty-three to one hundred twenty-eight electrodes) or intermediate density (approximately one hundred twenty-nine to two hundred fifty-six electrodes) spandex cap 41, in the preferred embodiment, such as that manufactured by Neuroscan Labs, Inc. of El Paso, Tex. Each electrode 51 is joined to cap 41 by a nonconductive electrode holder, such as the exemplary rubber grommet 63 and O-ring 64 combination illustrated in FIG. 10.

Electrodes 51 may be formed from tin, silver, silver chloride, sintered materials, platinum, gold or any other suitable conductor. Each electrode is preferably numbered or otherwise marked with indicia on or near the electrode lead 55. Alternatively, the electrodes 51 may be formed from glassy carbon materials to provide MRI/CT transparency.

Alternatively, the electrodes 51 may be interconnected with fiber optic leads in order to decrease the weight of the harness, increase patient comfort and provide easier and more efficient manipulation of support 25. In this optic-based embodiment, an amplifier is provided for amplifying signals generated by the patient. The amplified signal is passed to an analog-to-digital converter, and the digital signal is sent to an optical transmitter. The optical transmission is transmitted via the fiber optics to an optical receiver, which is in communication with a preamplifier. The signal is converted back into an electrical signal, processed and then transmitted to an amplifier. The amplified signal is then transmitted to a workstation for processing and analysis. Fiber optics, optoelectronic systems and components for scientific and medical uses are known. Such systems are manufactured by, for example, the Optical Corporation of America, of Marlborough, Mass.

In the preferred embodiment of FIG. 10, leads 55 are formed from carbon fiber materials. Alternatively, leads 55 may be replaced by a series of wireless transmitters and receivers. As a further alternative, the electrode assembly 51 may be replaced by a subdermal needle electrode. Such an electrode may be applied to the scalp with electronic, pre-programmed or computer-controlled placement. Further, a safety collar may be placed on the needle electrode assembly so that the needle penetrates the epidermis of the scalp to an exact depth, thus minimizing injury to the surrounding tissue. Alternatively, wireless disposable scalp, truck and limb electrodes for SEPs or other desired studies may replace the subdermal disposable needle electrodes.

In use, an electrode cap 41 containing sixty-four electrodes, for example, with the electrodes being filled with conductive gel simultaneously, can be fully filled with gel in a few minutes. After this initial filling, two technicians, for example, can abrade the scalp as needed and reduce impedance to acceptable levels within a few additional minutes. A high-density electrode cap containing 1,536 electrodes, which approaches full coverage of the average adult scalp, can be filled within a few minutes. The scalp can subsequently be abraded within an estimated time of approximately two hours.

FIG. 11 illustrates a MEG electrode assembly similar to the EEG assembly 51 of FIG. 10. The MEG sensors 810 are mounted on cap 41 in a manner similar to the mounting of EEG sensors 51. Each MEG sensor 810 includes at least one independent reference magnetic field sensor to reduce environmental noise to acceptable levels, and a plurality of suitable independent primary magnetic field sensors capable of detecting magnetic energy emanating from spontaneous and evoked neuronal signals in the human brain.

The sensor 810 includes a high order gradiometer detection coil 720 and a magnetic input coil 730. Further, a superconductive quantum interference device (SQUID) sensor is preferably provided, thus requiring the input of liquid coolant, such as liquid nitrogen 670. Liquid coolant 670 is fed into the sensor assembly 810 via port 680 in a manner similar to the pumping of conductive gel in the embodiment of FIG. 10. Similarly, a harness lead 700 is provided, mounted within an insulating lead support 690. The sensor assembly is contained within an insulating housing 710.

The positioning of the magnetic field sensors adjacent the scalp 71 allows for the detection of magnetic fields generated by brain neuronal activity. The detection coil 720 senses changes in the magnetic field and transforms them into an electrical current 47. Input coil 730 transforms this current into a magnetic flux within the sensor. The sensed signal is transposed unidirectionally via a magnetic field across a thin dielectric film 77, where it then amplified and conditioned with suitable integrated electronic circuits, as described above (indicated by diagrammatic arrow 79), to produce an isolated replica of the input signal 49. The plurality of simultaneously measured channels are amplified and transmitted independently via suitable harness leads 700 to suitable electronic information processors housed within housing 13.

Alternatively, magnetic sensors may be selected that do not require the input of liquid coolant. Further, the sensors may be superconductors, conventional metals, or a combination of the two. Additionally, system 10 may include a cap 41 including both EEG sensors 51 and MEG sensors 810.

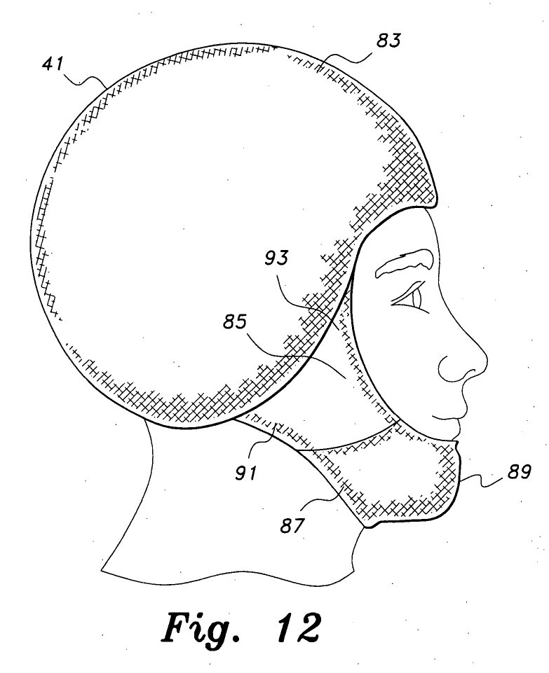

A preferred electrode cap 41 is illustrated in FIG. 12. Cap 41 is preferably seamless and is woven with elastic thread or cord 83. The seamless nature of cap 41 provides for a better fit, and thus better contact, on the patient's scalp. By positioning the electrodes 51 flush against the patient's scalp, without unnecessary spacing therebetween, the number of false signals produced is greatly reduced.

The seamless nature of cap 41 further allows a relatively high number of sensors to be mounted to the cap, thus increasing the amount of data an encephalographic system may obtain during a measurement session. In manufacture, electrode locations are preferably calculated and marked on a uniform template model, to be used in the production of each cap. The electrode number and template selected may vary according to cap size and application.

Electrode cap 41 is held to the patient's head by a mandibular strap 85, which includes a stretchable fabric extending bilaterally from the mandibular angle 87 of the patient, posteriorly throughout the base of the mandible, to the protuberance 89 of the mandible anteriorly. The mandibular strap has a contour so that it covers and extends from the mandibular ramus 91 to the zygomatic arch 93. The strap 85 is preferably approximately five to eight inches wide. The strap 85 may further include a selectively releasable and adjustable fastener, such as a hook and loop type fastener, for example.

Preferably, the strap 85 is worn to be taut across the entire anterior portion of the patient's chin, thus allowing the force required to maintain cap 41 on the patient's head to be distributed across the length of the mandible, rather than just at the mental protuberance portion of the chin.

The cap 41 may, alternatively, be formed of latex rubber, plastic or any other suitable elastic compound, and is formed with a plurality of openings formed therethrough for the mounting of single-use or repeat-use electrodes and sensors. An elastic mold may be taken of the patient's head for production of a custom-fit cap 41, which may be available for single-session usage, or may be used multiple times with the same patient.

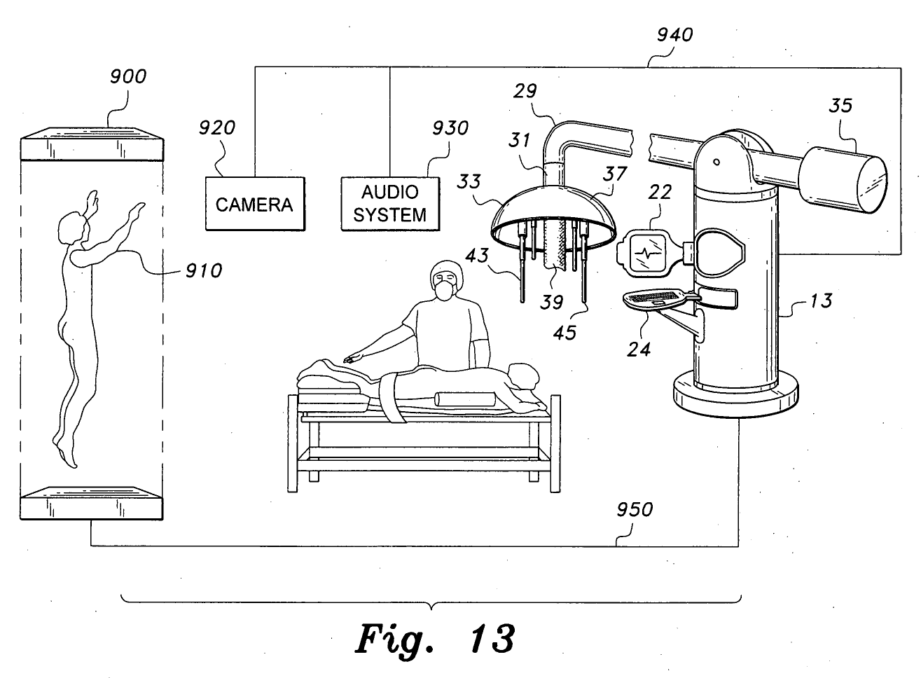

In the alternative embodiment of FIG. 13, system 10 utilizes a holographic display device 900 to monitor the patient during a neurological procedure. The holographic display device 900 generates a three-dimensional holographic image 910 of the patient for the medical practitioner to view and refer to before, during or after the procedure. In conventional photographic holography, the recording medium is typically film, so that the hologram image is permanently formed. This static recording is similar to a photograph, which is also a permanent recording of image information. Thus, once formed, the hologram is “fixed,” or unchangable.

In contrast, display 900 utilizes a dynamic hologram, which consists of electromagnetic stimulation of, preferably, a nonlinear optical material. Such materials may include photorefractive crystals, atomic vapors and gases, semiconductors (including quantum well devices), plasmas and some liquids. The local absorption and/or phase in the nonlinear material is exposed during stimulation and tracks changes in the interference pattern formed by the recording light beams. As the interference pattern changes, the local absorption and/or phase pattern in the material will also change and replace the original pattern.

Beyond these passive materials, the dynamic media can also be in the form of active electro-optical devices, such as spatial light modulators (SLMs), for example. In this case, the pixelated image-bearing input port serves as the dynamic recording material, whereas the pixelated output of the device (e.g., the output display, or projection port) functions as the effective holographic reconstruction port. SLMs may include the use of liquid crystal layers as well as micro-electrical mechanical (MEMS) technologies as the pixelated image-bearing output (projection) port. The pattern imposed onto the input port of the SLM will give rise to a corresponding output pattern, as read out by the reconstruction beam. By virtue of the SLM, the output, or reconstruction, beam will be spatially encoded as a corresponding amplitude, phase or polarization pixelated mapping of the input image. It should be understood that any three-dimensional dynamic holographic display may be utilized, including, for example, holographic tanks and split LCD displays, which transmit separate images to each eye of the user.

Further, the dynamic holography system may utilize wave front reconstruction, ultrasonic wave holography or any other suitable holographic technique. Further, the holographic display unit can be coupled with microscopy, which is of particular use in reference to medical procedures. The holographic system of FIG. 13 may provide holographic images including displays representing the brain, the spinal cord and other tissue of interest, pre-procedure, during the procedure and post-procedure.

In FIG. 13, the image of the patient is recorded by camera 920, which may be a videocamera, or the like. The camera input is fed, via line 40, in the computer system housed within housing 13, for processing into holographic display data. This data is fed, via line 950, to the holographic display unit 900 to produce image 900.

Further, audio system 930 includes an audio input device, such as a microphone, and an audio output device, such as a speaker. During the medical procedure, pre-recorded messages, as reminders or warnings of medical hazards, can be delivered to the medical practitioner through audio system 930. Further, the input of both camera 920 and audio system 930 allows for the medical practitioner to interact with the holographic image 910; i.e., through the use of speech, pattern and movement recognition, the medical practitioner's movements, speech and gestures can be interpreted by the computer system housed within housing 13 to generate responses, in the form of speech or movement, associated with the display image 910. Glow-in-the-dark, sterile or non-sterile, latex or latex-free gloves for use in medical settings are further provided for the surgeon or medical professional to use in communication with the holographic image.

In addition to the video image recorded by camera 920, the holographic display image 910 may include further data gathered from medical sensing procedures, such as those described in detail above with specific relation to system 10, so that the image 910 may display the internal organs of the patient, the skeleton of the patient, the brain and brain mapping function, or any other desired portion of the patient's anatomy, and in any desired proportions. This dynamic medical display of the patient may be used as a tool during the performance of a procedure, as a reference guide, as a diagnostic tool or for teaching purposes.

Further, a holographic display system, such as system 900, along with audio and video systems, such as systems 930, 920, could be located outside of the medical center housing the patient; i.e., the holographic display 910 can be viewed via telepresence communication links at a separate location, with the ability for viewers to interact with the image 910 in the same manner as the interaction between the image 910 and the medical practitioner. Further, for purposes such as teaching, for example, a selected field of view may be provided to remote viewers, allowing the viewers to observe the medical practitioner or a selected portion of the practitioner in addition to the image of the patient.

Utilizing system 10, the neurological and physiological data associated with a particular medical test is stored, and may be incorporated with display image 910. For example, when stimulating a peripheral nerve or a pedicle screw, the holographic image 910 may display and replay (at a selected speed), the electrical current as it travels proximally to the spinal cord. If the nerve is compressed, an animated light burst, for example, may appear holographically at that level in the cord, alerting the medical practitioner to the problem. The visual current would then, for example, proceed up the spinal cord, to the brain stem and into the brain, or down the spinal cord and through the neuromuscular junction.

The holographic display system may further provide three dimensional playback of stored holographic imagery, in addition to the real-time display system described above. Further, training simulations and illustrations may be pre-recorded and displayed using the holographic imagery system for both training purposes and also to be used as a guide tool and reference during a medical procedure. The holographic images may include additional waveforms, graphs, charts, text and iconography, such as arrow pointers, for example, for training and reference purposes. Additionally, the image may be selectively translated, rotated, angularly adjusted, enlarged or decreased in size, played over a selected time increment, played at a selected speed, or at least partially made transparent, allowing the medical practitioner to view only selected portions of the patient's anatomy. The holographic display system may be used in combination with data collected via MRI, fMRI, CT scans, EEGs, MEGs, ventricular studies, PET scans, SPECT scans, sonography, TBM, electrocochleography, deep brain stimulation, brain stem auditory evoked response tests, visual evoked potentials, SEPs, NCS, RNS, F-wave and H-reflex studies, surface and needle EMGs, MEPs, PSG, TCD, voxel-based lesion symptom mapping (VLSM) and any other suitable or desired tests associated with equipment 15.

It should be understood that a similar display system may be used with conventional display technology; i.e., a virtual patient may be generated on a conventional two-dimensional display, such as display 22 mounted on housing 13, and the medical practitioner can interact with the virtual patient, as described above, via the video and audio systems 920, 930, and with user-interface 24, which may be a keyboard or the like. Further, video monitoring may be provided for use with surgical microscope or other suitable desired operative and clinical instrumentation.

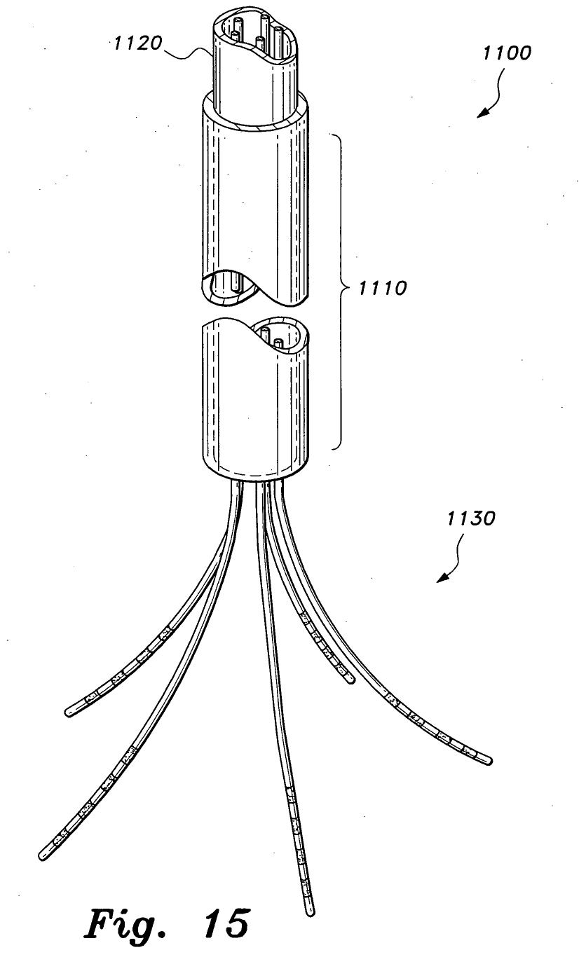

Further, FIG. 15 illustrates a “flower-spray” electrode 1100, for use in microelectrode mapping (MEM) for the deep brain stimulation (DBS) component of the testing and measuring system. Electrode 1100 allows for a sensor “spray” or spread toward specific somatotopically arranged nervous tissue. Electrode 1100 includes an outer layer of cladding or insulation 1110, for receiving cable or wire bundle 1120. Multiple lead extensions 1130 “spray” or fan out to a larger area than in conventional single-extension electrodes, thus stimulating the intended somatotopic area to an enhanced degree. Each individual extension 1130 can be tested individually and turned on or off selectively, as desired. The extensions 1130 can further be provided in multiple lengths, and having a variety of contours, such as ends shaped as balls, cups, etc., depending upon the particular needs and desires of the user.

It is to be understood that the present invention is not limited to the embodiments described above, but encompasses any and all embodiments within the scope of the following claims.

I claim:

1. A neurophysiology testing and monitoring system, comprising:
a housing having an upper end and a lower end, the housing defining an open chamber;
a neurological measurement system disposed within the chamber defined by housing;
an elongated support having a proximal end and a distal end, the proximal end being pivotally and adjustably mounted on the upper end of the housing;
a sensor cap mounted on the distal end of the elongated support, the sensor cap being adapted for positioning on the head of a user; and
at least one sensor mounted on the sensor cap and projecting downwardly from an inner surface thereof, the sensor being adapted for contacting the head of the user, the at least one sensor being in electrical communication with the neurological measurement system, whereby the elongated support may be selectively positioned so that the distal end of the elongated support is positioned adjacent the head of the user.

2. The neurophysiology testing and monitoring system as recited in claim 1, further comprising a holographic display system in electrical communication with said neurological measurement system for displaying at least a partial three-dimensional view of the anatomy of the user.

3. The neurophysiology testing and monitoring system as recited in claim 1, wherein said at least one sensor comprises an electrode having an upper end and a lower end, the lower end forming an electrode cup adapted for mounting on the head of the user, the electrode cup defining an inner open region, the upper end having a gel port formed therethrough, the gel port being in fluid communication with the inner open region, the system further comprising:
a conductive gel supply disposed within said housing; and
means for selectively and adjustably dispensing conductive gel from the conductive gel supply.

4. The neurophysiology testing and monitoring system as recited in claim 1, wherein said neurological measurement system is selected from the group consisting of: an electroencephalogram system, a magnetoencephalogram system, a surface electromyography system, a needle electromyography system, a motor nerve conduction test system, a sensory nerve conduction test system, a somatosensory evoked potential system, a repetitive nerve stimulation study system, a brainstem auditory evoked response system, a visual evoked potentials system, a polysomnography system, a ventriculography system, a Doppler study system, an H-reflex system, an F-wave system, a spinal cord monitoring system, a brain and spinal cord mapping system, a cognitive study system and a deep brain stimulation system.

5. The neurophysiology testing and monitoring system as recited in claim 1, further comprising a crown mounted on the distal end of said elongated support, the crown having an upper surface and a lower surface, said sensor cap extending downwardly from the lower surface.

6. The neurophysiology testing and monitoring system as recited in claim 5, further comprising a harness adapted to be releasably worn by the user, the harness being mounted to the lower surface of the crown and projecting downwardly therefrom, the harness being joined to, and supporting, said sensor cap.

7. The neurophysiology testing and monitoring system as recited in claim 5, further comprising at least one extendible arm mounted to the lower surface of the crown and extending downwardly therefrom, the at least one extendible arm being adapted for adjustably supporting auxiliary neurological testing equipment adjacent the user's head.

8. The neurophysiology testing and monitoring system as recited in claim 1, further comprising means for selectively driving and positioning said elongated support.

9. The neurophysiology testing and monitoring system as recited in claim 3, wherein the electrode is selected from the group consisting of: an electroencephalogram electrode and a magnetoencephalogram electrode.

10. The neurophysiology testing and monitoring system as recited in claim 1, wherein the neurophysiology testing and monitoring system is portable.

11. An electrode for use with a neurophysiology testing and monitoring system, comprising:
an electrode cup having an upper end and a lower end, the lower end being open and being adapted for placement on the head of a user, the upper end having an opening formed therethrough;
an elongated shaft mounted on the upper end of the electrode cup and extending upwardly therefrom, the elongated shaft having an inner chamber defined therein, a sidewall of the elongated shaft having a fluid port formed therethrough, the inner chamber being in fluid communication with an inner region of the electrode cup through the opening; and
an electrical lead mounted on the elongated shaft adapted for electrical connection to an external neurological measurement system, whereby an external fluid supply may be selectively connected to the fluid port for selective application of the fluid to the electrode cup.

12. The electrode for use with a neurophysiology testing and monitoring system as recited in claim 11, wherein an upper end of said elongated shaft is open and adapted for receiving an abrading instrument, whereby said abrading instrument may be inserted through said upper end of said elongated shaft to pass into said electrode cup through the opening to abrade the head of the user prior to application of the fluid.

13. The electrode for use with a neurophysiology testing and monitoring system as recited in claim 11, wherein the electrode cup is adapted for electroencephalography.

14. The electrode for use with a neurophysiology testing and monitoring system as recited in claim 13, wherein the fluid is electrically conductive gel.

15. The electrode for use with a neurophysiology testing and monitoring system as recited in claim 11, wherein the electrode cup is adapted for magnetoencephalography.

16. The electrode for use with a neurophysiology testing and monitoring system as recited in claim 15, wherein the fluid is a coolant.

17. The electrode for use with a neurophysiology testing and monitoring system as recited in claim 15, further comprising a magnetic field sensor.

18. A neurophysiology testing and monitoring system, comprising:
a housing having an upper end and a lower end, the housing defining an open chamber;
a neurological measurement system disposed within the chamber defined by housing;
an elongated support having a proximal end and a distal end, the proximal end being pivotally and adjustably mounted on the upper end of the housing;
a sensor cap mounted on the distal end of the elongated support, the sensor cap being adapted for positioning on the head of a user;
at least one sensor mounted on the sensor cap and projecting downwardly from an inner surface thereof, the sensor being adapted for contacting the head of the user, the at least one sensor being in electrical communication with the neurological measurement system;
means for recording a visual representation of the user; and,
means for selectively displaying the visual representation of the user and user-selectable neurological measurement data, whereby the elongated support may be selectively positioned so that the distal end of the elongated support is positioned adjacent the head of the user.

19. The neurophysiology testing and monitoring system as recited in claim 18, wherein said means for selectively displaying the visual representation of the user and user-selectable neurological measurement data comprises a holographic display system.

20. The neurophysiology testing and monitoring system as recited in claim 18, further comprising:
means for recording audio signals coupled to said means for recording a visual representation of the user; and,
means for interpreting voice commands generated by the user.
